(19) United States
(12) Patent Application Publication
(10) Pub. No.: US 2005/0079201 A1
(43) Pub. Date: Apr. 14, 2005

(54) Implants with functionalized carbon surfaces

(76) Inventors: Jorg Rathenow, Wiesbaden (DE); Soheil Asgari, Munchen (DE); Andreas Ban, Koblenz (DE); Jurgen Kunstmann, Bad Soden (DE); Bernhard Mayer, Mainz (DE)

Correspondence Address: FROMMER LAWRENCE & HAUG, 745 FIFTH AVENUE- 10TH FL., NEW YORK, NY, 10151, US

(21) Appl. No.: 10/939,021

(22) Filed: Sep. 10, 2004

Related U.S. Application Data
(63) Continuation-in-part of application No. PCT/EP04/05785, filed on May 28, 2004.

(30) Foreign Application Priority Data
May 28, 2003 (DE) 103 24 415.8
Jul. 21, 2003 (DE) 103 33 098.4
Jul. 21, 2003 (DE) 103 33 099.2

(51) Int. Cl. B05D 3/04; A61F 2/02; B05D 3/10

(52) U.S. Cl. 424/424; 623/23.74; 424/426; 427/2.21; 427/2.24

(57) ABSTRACT

The invention relates to a method of producing medical implants having functionalized surfaces by providing a medical implant with at least one carbon-based layer on at least one part of the surface of the implant, activating the carbon-based layer by creating porosity and functionalizing the activated carbon-based layer. This invention also relates to functionalized implants obtained in by this method.

### INCORPORATION BY REFERENCE

This application is a continuation-in-part application of international patent application Serial No. PCT/EP2004/005785 filed May 28, 2004, which claims benefit of German patent application Serial Nos. DE 103 24 415.8 filed May 28, 2003; DE 103 33 098.4 filed Jul. 21, 2003 and DE 103 33 099.2 filed Jul. 21, 2003.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The present invention relates to a method for producing medical implants having functionalized surfaces by providing a medical implant with at least one carbon-based layer on at least a portion of the surface of the implant, activating the carbon-based layer by creating porosity and functionalizing the activated carbon-based layer; this invention also relates to functionalized implants obtainable by this method.

### BACKGROUND OF THE INVENTION

Medical implants such as surgical and/or orthopedic screws, plates, joint prostheses, artificial heart valves, vascular prostheses, stents as well as subcutaneously or intramuscularly implantable active agent depots are produced from a wide variety of materials, which are selected according to specific biochemical and mechanical properties. These materials must have certain mechanical and biochemical properties, must not release any toxic substances and must be suitable for long-term use in the body.

However, the metals or metal alloys and ceramic materials frequently used for stents and joint prostheses, for example, often have disadvantages with regard to their biocompatibility or functionality, in particular in long-term use. Implants trigger inflammatory tissue responses and immune reactions through chemical and/or physical irritation, thus resulting in intolerance reactions in the sense of chronic inflammatory reactions with defensive and rejection reactions, excessive scar tissue production or degradation of tissue, which in the extreme case must result in the implant having to be removed and replaced or additional therapeutic interventions of an invasive or noninvasive type being indicated.

For this reason, there are various approaches in the state of the art for coating surfaces of medical implants in a suitable way to increase the biocompatibility of the materials used or the functional efficacy of the implants and to prevent defensive reactions, i.e., rejection.

U.S. Pat. No. 5,891,507, for example, discloses methods for coating the surface of metal stents with silicone, polytetrafluoroethylene and biological materials such as heparin or growth factors which increase the biocompatibility of the metal stents.

In addition to plastic layers, carbon-based layers have proven to be particularly advantageous.

For example, German Patent DE 199 51 477 describes coronary stents with a coating of amorphous silicon carbide, which increases the biocompatibility of the stent material. U.S. Pat. No. 6,569,107 describes carbon-coated stents in which the carbon material is applied by chemical or physical vapor-phase deposition methods (CVD or PVD). U.S. Pat. No. 5,163,958 also describes tubular endoprostheses or stents with a coated surface having antithrombogenic properties. WO 02/09791 describes intravascular stents with coatings produced by CVD of siloxanes.

In addition to the CVD methods for deposition of carbon, various high-vacuum sputtering methods have been described in the state of the art for production of pyrolytic carbon layers with various structures (see U.S. Pat. No. 6,355,350, for example).

The implants with modified surfaces produced in this way still have some disadvantages, however. The biocompatibility is not adequate in all cases to completely prevent rejection reactions. Furthermore, the surface-coated implants of the state of the art usually have closed pores, which make intergrowth with the surrounding body tissue difficult or impossible and/or restrict functionalization. Although the prior art implants can also be coated with antibiotics, the effect of the substances after implanting the implant is always short lasting, however, because the quantities of active ingredient applied are limited by the nature of the implant and by its surface coating or its desorption cannot be controlled or its efficacy is impaired by physical or chemical interactions with the coating.

Furthermore, it is appropriate and advisable from a medical standpoint if implants can be used not only in their supporting function, as is the case with stents, but can also be provided with additional functions, e.g., long-term delivery of medications at the site of implantation of the implant to potentiate the effect of the implant or to achieve additional medically desirable effects.

There has therefore been a demand for inexpensive, easy-to-use methods for producing functionalized implants.

Furthermore, there has also been a demand for medical implants that are inexpensive to manufacture and have improved properties.

Citation or identification of any document in this application is not an admission that such document is available as prior art to the present invention.

### SUMMARY OF THE INVENTION

One object of the present invention is therefore to make available a method for producing implants with an additional functionality.

Another object of the present invention is to make available medical implants which can assume additional functions, such as the release of pharmaceutical substances in the body or the growth of tissue, and thereby have increased biocompatibility and/or have a stronger functional implant effect.

Another object of the present invention is to make available medical implants which permit a long-term release of medical active ingredients in the body of the patient or which have a function that is improved by surface modification.

Yet another function of the present invention is make available medical implants which can release pharmacologically active substances that are applied to or incorporated into the implant in a controlled manner after implantation of the implant in the human body.

Another object of this invention is to provide implantable active ingredient depots with a coating which is capable of controlling the release of active ingredients from the depot.

Another object of this invention is to make available medical implants which contain applied and/or incorporated microorganisms, viral vectors or cells or tissue, so that after the implant has been implanted in the human body, a therapeutic effect can be achieved in a control manner or the bioavailability can be increased.

The inventive solution to the objects indicated above consists of a method and medical implants obtainable by this method as defined in the independent claims. Preferred embodiments of the inventive method and/or the inventive products and uses are derived from the dependent subordinate claims.

Within the context of the present invention, it has been discovered that carbon-based layers in particular on implantable medical devices of a wide variety of types can be utilized easily to equip the implant with additional medical physiological and therapeutic functions.

It is possible in particular according to this invention to apply therapeutically active quantities of pharmaceutical agents to the surface of an implant or in a layer present on the implant and to release these substances continuously in a controlled manner in the human body.

Accordingly, the inventive method for producing medical implants having functionalized surfaces comprises the following steps:
a) providing a medical implant with at least one carbon-based layer on at least a portion of the surface of the implant;
b) activating the carbon-based layer by creating porosity;
c) functionalizing the activated carbon-based layer.

With the inventive method, it is possible to suitably modify implants having carbon-based surface coatings to make it possible to load them with therapeutically active amounts of active pharmacologic substances. By creating porosity in carbon-based surface layers of a suitable thickness, controlled adjustment/modification of the pore size and/or pore structure and optionally a suitable surface coating that modifies release, it is possible to adjust and vary in a controlled manner the load quantity, type and rate of release as well as the biological physiological surface properties. This also makes it possible to implement embodiments tailored to each specific type of implant and active ingredient as well as each site of application and intended use of medical implants with simple procedural measures such as those described according to this invention.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DETAILED DESCRIPTION

Implants coated with a carbon-based coating can be functionalized by the method according to this invention.

The terms “implantable medical device” and “implant” are used synonymously here and are understood to include medical or therapeutic implants, such as vascular endoprostheses, intraluminal endoprostheses, stents, coronary stents, peripheral stents, surgical and/or orthopedic implants for temporary use, such as surgical screws, plates, nails and other fastening means, permanent surgical or orthopedic implants, such as bone prostheses or joint prostheses, e.g., artificial hip or knee joints, socket joint inserts, screws, plates, nails, implantable orthopedic fixation aids, vertebral prostheses and artificial hearts and parts thereof as well as artificial heart valves, heart pacemaker casings, electrodes, subcutaneous and/or intramuscularly implantable implants, active ingredient depots and microchips and the like.

The implants that can be used in the inventive method may consist of almost any materials, preferably those that essentially have thermal stability, in particular all materials of which such implants are typically produced.

Examples include, but are not limited to, amorphous and/or (partially) crystalline carbon, solid carbon material, porous carbon, graphite, carbon composite materials, carbon fibers, ceramics such as zeolites, silicates, aluminum oxides, aluminosilicates, silicon carbide, silicon nitride, metal carbides, metal oxides, metal nitrides, metal carbonitrides, metal oxycarbides, metal oxynitrides and metal oxycarbonitrides of the transition metals, such as titanium, zirconium, hafnium, vanadium, niobium, tantalum, chromium, molybdenum, tungsten, manganese, rhenium, iron, cobalt, nickel; metals and metal alloys, in particular the noble metals such as gold, silver, ruthenium, rhodium, palladium, osmium, iridium, platinum; metals and metal alloys of titanium, zirconium, hafnium, vanadium, niobium, tantalum, chromium, molybdenum, tungsten, manganese, rhenium, iron, cobalt, nickel, copper; steel, in particular stainless steel, memory alloys such as nitinol, nickel titanium alloy, glass, stone, glass fibers, minerals, natural or synthetic bone substance, imitation bone based on alkaline earth metal carbonates such as calcium carbonate, magnesium carbonate, strontium carbonate, foamed materials such as polymer foams, foamed ceramics and the like as well as any combinations of the aforementioned materials.

In preferred embodiments of the present invention, the implants used are stents, in particular metal stents, preferably made of stainless steel, platinum-based radiopaque steel alloys, so-called PERSS (platinum-enhanced radiopaque stainless steel alloys), cobalt alloys, titanium alloys, high-melting alloys, e.g., based on niobium, tantalum, tungsten and molybdenum, noble metal alloys, nitinol alloys as well as magnesium alloys and mixtures of the above.

Especially preferred implants within the scope of the present invention include, but are not limited to, stents made of stainless steel, in particular Fe-18Cr-14Ni-2.5Mo (“316LVM” ASTM F 138), Fe-21Cr-10Ni-3.5Mn-2.5Mo (ASTM F 1586), Fe-22Cr-13Ni-5Mn (ASTM F 1314), Fe-23Mn-21Cr-1Mo-1N (nickel-free stainless steel); stents made of cobalt alloys such as Co-20Cr-15W-10Ni (“L605” ASTM F 90), Co-20Cr-35Ni-10Mo (“MP35N” ASTM F 562), Co-20Cr-16Ni-16Fe-7Mo (“Phynox” ASTM F 1058). Examples of preferred titanium alloys include, but are not limited to, CP titanium (ASTM F 67, Grade 1), Ti-6Al-4V (alpha/beta ASTM F 136), Ti-6Al-7Nb (alpha/beta ASTM F1295), Ti-15Mo (beta grade ASTM F 2066); stents made of noble metal alloys in particular alloys containing iridium such as Pt-10Ir; nitinol alloys such as martensitic, superelastic and cold-workable (preferably 40%) nitinols and magnesium alloys such as Mg-3Al-1Z.

The implantable medical devices that can be used according to this invention may have almost any external form. The inventive method is not limited to certain structures.

The implants must have a carbon-based layer on at least a portion of their surface. This layer may consist of pyrolytically produced carbon, vitreous amorphous carbon, vapor-deposited carbon, carbon applied by CVD, PVD or sputtering, diamond-like, graphitic carbon, metal carbides, metal carbonitrides, metal oxynitrides or metal oxycarbides as well as any combinations thereof. The carbon-based layer may be amorphous, partially crystalline or crystalline, preferably consisting of layers of amorphous pyrolytic carbon, and in some embodiments it may also be diamond-like, e.g., vapor-deposited carbon.

Implants with a carbon-based coating produced by applying material that produce carbon and/or polymer films to the implant and then carbonizing these materials in the absence of oxygen at elevated temperatures are especially preferred. Examples are disclosed in German Patent DE 10322187 and/or PCT/EP2004/005277, German Patent DE 10324415 and/or PCT/EP2004/004987 and German Patent DE 10333098 and/or PCT/EP2004/004985, the disclosures of which are herewith fully incorporated by reference.

Other suitable implants with a carbon-based coating include, but are not limited to, commercial carbon-coated implants such as metal stents of the Radix Carbostent® type (Sorin Biomedica) and the like, most of which have carbon coatings produced by physical vapor deposition or atomization methods, including sputtering.

The thickness of one or more carbon-based layers may in general be from 1 nm to 1 mm, optionally even several millimeters, e.g., up to 10 mm, preferably up to 6 mm, especially preferably up to 2 mm, in particular between 10 nm and 200 μm.

In preferred embodiments of the present invention, the implantable medical devices may also have multiple carbon-based layers of the same or different thickness and/or porosity. For example, it is possible to combine deeper more porous layers with narrow-pored layers above them which can suitably delay the release of the active ingredients deposited in the more porous layer.

According to the inventive method, the physical and chemical properties of the carbon-based coating are further modified by suitable activation steps and adapted to the intended purpose. Traditional carbon-based implants usually have essentially closed surfaces, which greatly restrict effective and long-lasting loading with active ingredients, for example, or limit it to a very small amount. The purpose of activation is to create porosity in the carbon-based layer and/or to form a porous carbon-based layer on the implant in order to thereby permit better functionalization by means of active ingredients, cells, proteins, microorganisms, etc., and to increase the ability to uptake the carbon-based layer per unit of area.

The activation step in the inventive method thus consists essentially of creating porosity in the carbon layer on the implant. Several possibilities are available here.

One possible method of activating the carbon layer includes, but is not limited to, for example, reductive or oxidative treatment steps in which the layer is treated one or more times with suitable reducing agents and/or oxidizing agents, such as hydrogen, carbon dioxide, water vapor, oxygen, air, nitrous oxide or oxidizing acids such as nitric acid and the like and optionally mixtures of these.

Activation with air is preferred, especially preferably at an elevated temperature.

The activation step(s) may be carried out at an elevated temperature, e.g., from 40° C. to 1000° C., preferably 70° C. to 900° C., especially preferably from 100° C. to 850° C., in particular preferably from 200° C. to 800° C. and most especially at approximately 700° C. In especially preferred embodiments, the carbon-based layer is modified by reduction or oxidation or a combination of these treatment steps at room temperature. Boiling in oxidizing acids or bases may also be used to produce a porous surface.

The pore size and pore structure can be varied according to the type of oxidizing agent or reducing agent used, the temperature and the duration of the activation. In particularly preferred embodiments, carbon-based medical implants activated according to this invention can be used for controlled release of active ingredients from the substrate into the environment through targeted adjustment of the porosity of the carbon layer.

The coatings are preferably porous after activation, in particular having a nanoporosity. Medical implants can be used as a drug vehicle with a depot effect according to this invention, for example, in particular when the implant itself also has a porous structure, in which the activated carbon-based layer of the implant can be used as a membrane which regulates the rate of release.

In preferred embodiments, the porosity can be adjusted by washing out fillers that are present in the carbon-based coating, e.g., polyvinylpyrrolidone, polyethylene glycol, powdered aluminum, fatty acids, microwaxes or emulsions, paraffins, carbonates, dissolved gases or water-soluble salts with water, solvents, acids or bases or by distillation or oxidative and/or non-oxidative thermal decomposition. Suitable methods of this are described in German Patent DE 103 22 187 and/or PCT/EP2004/005277, for example, by the present applicant, the disclosures of which are herewith fully incorporated by reference.

The porosity may optionally also be created by structuring the surface with powdered substances such as metal powder, carbon black, phenolic resin powder, fibers, in particular carbon fibers or natural fibers.

Another possibility for activation and/or producing porosity is by sputtering the carbon-based layer with suitable elements or so-called ion bombardment, e.g., with noble gas ions or the like.

The activated coating may optionally also be subjected to a so-called CVD process (chemical vapor deposition) or CVI process (chemical vapor infiltration) in another optional process step in order to further modify the surface structure or pore structure and its properties. To do so, the carbonized coating is treated with suitable precursor gases that release carbon at high temperatures. Subsequent application of diamond-like carbon is preferred here. Other elements may also be deposited in this way, such as silicon. Such methods are known in the state of the art.

Almost all known saturated and unsaturated hydrocarbons with sufficient volatility under CVD conditions may be used as the precursors to split off carbon. Examples include, but are not limited to, methane, ethane, ethylene, acetylene, linear and branched alkanes, alkenes and alkynes with carbon numbers of C₁ to C₂₀, aromatic hydrocarbons such as benzene, naphthalene, etc., and aromatics with one or more alkyl, alkenyl and alkynyl substituents, such as toluene, xylene, cresol, styrene, etc.

Suitable ceramic precursors include, but are not limited to, for example, BCl₃, NH₃, silanes such as SiH₄, tetraethoxysilane (TEOS), dichlorodimethylsilane (DDS), methyltrichlorosilane (MTS), trichlorosilyldichloroborane (TDADB), hexadichloromethylsilyloxide (HDMSO), AlCl₃, TiCl₃ or mixtures thereof.

In the CVD method, the precursors are mostly used in a low concentration of approximately 0.5 to 15 vol % in mixture with an inert gas such as nitrogen, argon or the like. Hydrogen may also be added to the corresponding gas mixtures for deposition. At temperatures between 500° C. and 2000° C., preferably 500° C. to 1500° C. and especially preferably 700° C. to 1300° C., the compounds mentioned above split off hydrocarbon fragments and/or carbon or ceramic precursors, which are deposited in an essentially uniform distribution in the pore system of the pyrolyzed coating, where they modify the pore structure and produce an essentially homogeneous pore size and pore distribution.

By means of CVD methods, the size of pores in the carbon-based layer on the implant can be reduced in a controlled manner or the pores may even be completely closed and/or sealed. This makes it possible to adjust the sorptive properties as well as the mechanical properties of the activated implant surface in a tailored manner.

By CVD of silanes or siloxanes, optionally in mixture with hydrocarbons, the carbon-based implant coatings can be modified by formation of carbide or oxycarbide, so that they are resistant to oxidation, for example.

In preferred embodiments, the coated implants activated according to this invention can be additionally coated and/or modified by sputtering methods. Carbon, silicon and metals and/or metal compounds can be applied by essentially known methods from suitable sputter targets. For example, by incorporating silicon compounds, titanium compounds, zirconium compounds, or tantalum compounds or metals by CVD or PVD into the carbon-based layer, it is possible to form carbide phases which increase the stability and oxidation resistance of the layer.

In another preferably embodiment of the activation step, carbon-based layers, even sputtered carbon layers, for example, can be worked mechanically afterwards to produce porous surfaces. For example, controlled abrasion of these layers by suitable methods leads to porous layers. A preferred option is abrasion of such carbon-based layers in an ultrasonic bath, where defects in the layers and thus porosity can be produced in a targeted manner by admixture of abrasive solids of various particle sizes and degrees of hardness by appropriate input of energy and a suitable frequency of the ultrasonic bath as a function of treatment time.

Aqueous ultrasonic baths to which alumina, silicates, aluminates and the like have been added, preferably alumina dispersions, are preferred here. However, any other solvents that are suitable for ultrasonic baths may also be used instead of or in combination with water.

For example, by treatment of carbon-coated implants in an aqueous ultrasonic bath with the admixture of alumina, preferably 1% to 60% alumina dispersions, it is possible to produce nano-abraded carbon layers with an average pore size of approximately 5 nm to 200 nm.

Furthermore, by ion implantation of metals, in particular transition metals and/or non-metals, the surface properties of the implant can be further modified. For example, by nitrogen implantation it is possible to incorporate nitrides, oxynitrides or carbonitrides, in particular those of the transition metals. The porosity and strength of the surface materials can be further modified by implantation of carbon.

The carbon-based layer is preferably porous after activation, with pore diameters in the range of 0.1 μm to 1000 μm, preferably between 1 μm and 400 μm. Macroporous layers can also be produced with the inventive activation steps.

The carbon-based layer is especially preferably nanoporous after activation with pore diameters of 1 nm to 1000 nm, preferably from 5 nm to 900 nm.

In an especially preferred embodiment of this invention, the activation is performed during the step of production of the carbon-based layer, e.g., by applying one or more porous carbon-based layers, by carbonization of substances that produce carbon, by coating with carbon by CVD or PVD and/or by applying suitable layers of porous biodegradable and/or resorbable or non-biodegradable and/or resorbable polymers.

It is especially preferable for one or more porous carbon-based layers to be applied by coating the implant with an optionally foamed polymer film or one containing fillers and then carbonizing the polymer film at temperature of 200° C. to 3500° C., preferably up to 2000° C. in an oxygen-free atmosphere, optionally partially oxidized in an air stream subsequently. Corresponding methods are described, for example, in German Patent DE 10324415 and/or PCT/EP2004/004987 and in German Patent DE 10333098 and/or PCT/EP2004/004985, the disclosures of which are herewith fully incorporated by reference.

Thus, for example, adding polyethylene glycol to the polymer film that is to be carbonized produces defects in the polymer crosslinking which in turn produce a porous carbon layer after thermal treatment or dissolving in suitable solvents. Porosity suitable for a given application can be achieved through the choice of the polymer system, the molecular weight of polyethylene glycol and the polyethylene glycol solids content, and in particular the average porosity, the pore size distribution and the degree of porosity can be adjusted. For example, by selecting polyethylene glycols with a molecular weight of 1000 to 8,000,000 Dalton, it is possible to produce pore sizes from 10 nm to 1000 nm or in a preferred embodiment from 50 nm to 1000 nm. By varying the solids content from 10% to 80%, a degree of porosity from 5% to 80% can be produced, preferably 20% to 60%.

Another example of this type of combined production and activation of the carbon-based layer is by admixture of carbon black to the polymer film. Through the choice of the average particle size and the solids content in the polymer film, it is possible to produce porous matrices in which the degree of porosity and average pore size can be adjusted through the choice of suitable polymer systems, carbon black particle sizes and the solids content, depending on the application. Thus, for example, by admixture of carbon black particles with an average particle size of 10 nm to 1 mm, preferably 10 nm to 1000 nm, with a solids content of 20% to 80%, preferably 30% to 60%, an average porosity of 30% to 60% can be produced, with the pore sizes produced being adjustable between 10 nm and 1000 nm, preferably from 10 nm to 800 nm.

Furthermore, by optional parylenation of the implants before or after the activation steps, the surface properties and porosity of the carbon-based layer can be further modified. The implants here are first treated with paracyclophane at an elevated temperature, usually approximately 600° C., with a polymer film of poly(p-xylylene) being formed on the surface of the implants. This film can then be converted to carbon by known methods in a subsequent carbonization step.

If necessary, in particularly preferred embodiments, the activated implant may be subjected to additional chemical and/or physical surface modifications. Cleaning steps to remove any residues and impurities that might be present may also be provided here. For this purpose, acids, in particular oxidizing acids, or solvents may be used, but boiling in acids or solvents is preferred. Carboxylation of these activated carbon layers can be achieved by boiling in oxidizing acids.

Before medical use or loading with active ingredients, the inventive implants may be sterilized by conventional methods, e.g., by autoclaving, ethylene oxide sterilization or gamma-radiation.

According to this invention, all possible activation methods may be combined or used with any of the functionalization steps described below.

The implants may be additionally equipped with a number of functions by suitable measures. Orthopedic and surgical implants or vascular endoprostheses may be used as drug vehicles or depots. The biocompatibility and functionality of the inventive implants can be influenced or altered in a controlled manner by incorporating additives, fillers, proteins, etc. This makes it possible to reduce or entirely prevent rejection reactions in the body when using implants produced according to this invention or the efficacy of the implant may be increased and/or additional effects achieved.

Functionalization in the sense of the present invention is understood to refer in general to measures as a consequence of which the carbon-based layer gains additional functions. Functionalization according to this invention consists of incorporating substances into the carbon-based layer or attaching substances to the carbon-based layer. Suitable substances are selected from pharmacological active ingredients, linkers, microorganisms, cells of plant or animal origin including human cells or cell cultures and tissue, minerals, salts, metals, synthetic or natural polymers, proteins, peptides, amino acids, solvents, etc.

According to this invention, the suitably activated implant can be functionalized by making it more biocompatible before or after a possible loading with active ingredients. This is done by coating it with at least one additional layer of biodegradable and/or absorbable polymers such as collagen, albumin, gelatin, hyaluronic acid, starch, celluloses such as methyl cellulose hydroxypropylmethyl cellulose, carboxymethyl cellulose phthalate; casein, dextrans, polysaccharides, fibrinogen, poly(D,L-lactides), poly(D,L-lactide-co-glycolides), poly(glycolides), poly(hydroxybutylates), poly(alkyl carbonates), poly(orthoesters), polyesters, poly(hydroxyvaleric acid), polydioxanones, poly(ethylene terephtalate), poly(malic acid), poly(tartronic acid), polyanhydrides, polyphosphazenes, poly(amino acids) and their copolymers or non-biodegradable and/or absorbable polymers. In particular anionic, cationic or amphoteric coatings are especially preferred, such as alginate, carrageenan, carboxymethyl cellulose, chitosan, poly-L-lysine and/or phosphorylcholine.

In the functionalization step of the inventive method, active ingredients such as pharmaceutical drugs and medications may be applied to the activated carbon-based layer or incorporated into the layer. This is useful in particular in cases where active ingredients cannot be applied in or to the implant directly as in the case of metals, for example.

For example, sparingly water-soluble lipophilic active ingredients such as paclitaxel are difficult to apply to metallic surfaces because they tend to form a crystalline film. Usually the immobilizable quantities are limited and the release cannot be controlled. Direct coating of such metallic surfaces with paclitaxel leads to maximum loading of approximately 3 mg/mm², the release of which under physiological conditions in physiological buffer solutions leads to uncontrolled desorption of max. 30% with in one to five days.

Carbon layers activated according to this invention, preferably vitreous amorphous carbon with a layer thickness in the range of 80 nm to 10 μm, preferably 100 nm to 5 μm, preferably with a pore size of 5 nm to 100 μm, preferably from 5 nm to 1000 nm can take up active ingredient quantities amounting to up to 100 times that of non-activated carbon-coated or purely metallic implants e.g., even at porosities of 5 to 50%, preferably 10 to 50% and an average pore size of 5 nm to 1 μm, preferably from 5 nm to 500 nm, and can optionally release these active ingredients in a controlled manner as a function of the porosity and/or pore size and/or surface properties.

In an inventive embodiment with a pore size of 50 nm and a porosity of 5%, for example, and with a load of 0.5 to 3.0 μmm² paclitaxel and hydrophobic carbon surfaces with a layer thickness of 200 nm, 70% to 100% of the applied dosage of paclitaxel can be released in a controlled manner at a constant daily release rate under physiological conditions within 25 to 35 days.

In particularly preferred embodiments, through suitable functionalization of any carbon-based layer, peptides and proteins as well as glycoproteins and lipoproteins can also be immobilized.

An inventive form of functionalization consists of covalent or non-covalent adsorption of substances which allow the binding of peptides, proteins, glycoproteins or lipoproteins labeled or otherwise provided with affinity tags.

Such substances include, but are not limited to, for example, ions, cations, in particular metal cations such as cobalt, nickel, copper and zinc cations, antibodies, calmodulin, chitin, cellulose, sugars, amino acids, glutathione, streptavidin, Strep-Tactin or other mutants for binding Strep-tag- or SBP-tag-labeled substances or S protein for binding S-tag-labeled substances, etc.

These affinity tags are attached by a suitable method to the peptides, proteins, glycoproteins or lipoproteins to be immobilized, attaching the either the C terminus or N terminus of the primary sequence, usually by the methods of recombinant genetic engineering or biotinylation. Affinity tags, in particular polyarginine tags (Arg tag) are preferred, the latter preferably consisting of five to six argininic acids, polyhistidine tags (His tags), a polyhistidine sequence of any desired length, typically two to ten radicals, FLAG tags with the sequence DYKDDDDK, Strep tags, e.g., the Strep tag II sequence WSHPQFEK, S tags which carry the amino acid radicals KETAAAKFERQHMDS, calmodulin-binding peptide, the family of cellulose-binding domains, in particular C-terminal, N-terminal or other positions in the primary sequence of the peptide, protein, glycoprotein or lipoprotein to be immobilized, the SBP tag with the sequence MDEKTTGWRGGHVVEGLAGELEQLRARLEHHPQGQREP, the polyhistidine tag, chitin-binding domains, glutathione S-transferase tag, maltose-binding protein, bacteriophage T7 and V5 epitope, but also any other type of affinity tag.

The modification of the substances to be applied to the functionalized carbon surfaces corresponds to the usual methods that are possible in purification and in particular chromatographic labeling.

Functionalization of the carbon surface is accomplished here by adsorption of corresponding substances in and/or on the carbon-based layer such that these substances can enter into a bond with the affinity tags. Corresponding substances include, but are not limited to, for example, cations which are introduced into the carbon layer to permit binding to the basic polyarginine tag, e.g., cobalt, nickel, copper and zinc cations to permit binding of polyhistidine tags, for example.

Adsorption of the antibody M1 on the carbon surfaces permits the binding of FLAG tags, streptavidin or Strep-Tactin or SBP tag-labeled substances or adsorption of the S protein on the surface to bind S tag labeled substances.

In another embodiment, the functionalization consists of using calmodulin which is to be adsorbed on the carbon surface. This makes it possible to bind calmodulin-binding peptide-labeled substances to the carbon-based layer.

In other embodiments, the functionalization is accomplished by adsorption of cellulose, so that substance modified with cellulose binding domains can be bound or it is accomplished by adsorption of chitin to bind substances provided with chitin-binding domains.

Similarly, functionalization may be performed with glutathione for binding glutathione S-transferase tag-labeled substances, or with maltose or amylose to bind maltose binding protein labeled substances.

Those skilled in the art will select a suitable affinity system in accordance with the conditions that are possible in genetic engineering, the functional and structural properties of the peptide, protein, glycoprotein or lipoprotein.

For example, carbon layers functionalized with 0.1-8 μg/mm² adsorbed Strep-Tactin can be obtained from a Strep-Tactin solution on porous carbon surfaces with a pore size of 100 to 900 nm, a porosity of 30% to 60% and a layer thickness of 1 to 5 μm by spraying or dipping carbon layers. The carbon layer functionalized in this way can take up, for example, 0.1 to 10 μg/mm² Strep-tag-labeled recombinant IL-2.

In another embodiment, the carbon layer is doped with cobalt ions, where the porous carbon matrix has a cobalt ion content of 0.1 to 50% of the solids content, preferably up to 60% in vitreous porous carbon layers. At a porosity of 50%, layer thickness of 500 nm to 1000 nm, 0.1 to 100 μg polyarginine tag-labeled recombinant IL-2 can be adsorbed by the metal ion doping in the matrix.

Another embodiment involves, for example, functionalization of the carbon surfaces by adsorption of linker substances, preferably carboxymethylated dextrans, e.g., as a hydrogel, which permits physical binding of substances, preferably biomolecules or active ingredients and/or have a chemical reactivity so that such substances can be attached by covalent bonds, preferably by forming amino, thiol or aldehyde bonds.

Those skilled in the art will select a suitable type of linker as a function of the type of ligand.

For the production of an amino bond, the carbon layer can be functionalized as follows in a preferred embodiments: adsorption of carboxymethylated dextran, subsequent modification by modification in NHS/EDC to convert the carboxymethyl groups into hydroxysuccinimide esters.

This makes it possible to adsorb ligands which enter into covalent amino bonds with the esters. Unreacted esters can be inactivated again in another step, e.g., by incubation in 1M ethanolamine hydrochloride solution. For example, adsorption of 1 μg carboxymethylated dextran per mm² of a porous carbon-based composite layer of vitreous carbon and carbon black particles yields a functionalization which can bind 0.01 to 5000 μg/mm² peptides with a molecular weight of 60 to 90 by covalent bonding.

Furthermore, the porous layers activated according to this invention can be loaded with pharmaceutical drugs, i.e., medications, microorganisms, cells and/or tissues in the functionalization step of the process or they may be provided with diagnostic aids such as markers or contract media for localizing coated implants in the body, e.g., even with therapeutic or diagnostic quantities of radioactive substances.

In preferred embodiments, the implants activated according to this invention are loaded with active ingredients in the functionalization step. Active ingredients may be loaded into or onto the carbon-based layer by suitable sorptive methods such as adsorption, absorption, physisorption or chemisorption; in the simplest case, they may be loaded by impregnation of the carbon-based coating with active ingredient solutions, active ingredient dispersions or active ingredient suspensions in suitable solvents. Covalent or non-covalent bonding of active ingredients in or on the carbon-based coating here may be a preferred option, depending on the active ingredient used and its chemical properties.

In preferred embodiments, the active ingredient is applied in the form of a solution, dispersion or suspension in a suitable solvent or solvent mixture, optionally with subsequent drying. Suitable solvents include, but are not limited to, for example methanol, ethanol, n-propanol, isopropanol, butoxydiglycol, butoxyethanol, butoxyisopropanol, butoxypropanol, n-butyl alcohol, t-butyl alcohol, butylene glycol, butyloctanol, diethylene glycol, dimethoxydiglycol, dimethyl ether, dipropylene glycol, ethoxydiglycol, ethoxyethanol, ethylhexanediol, glycol, hexanediol, 1,2,6-hexanetriol, hexyl alcohol, hexylene glycol, isobutoxypropanol, isopentyldiol, 3-methoxybutanol, methoxydiglycol, methoxyethanol, methoxyisopropanol, methoxymethylbutanol, methoxy PEG-10, methylal, methyl hexyl ether, methylpropanediol, neopentyl glycol, PEG-4, PEG-6, PEG-7, PEG-8, PEG-9, PEG-6-methyl ether, pentylene glycol, PPG-7, PPG-2-buteth-3, PPG-2 butyl ether, PPG-3 butyl ether, PPG-2 methyl ether, PPG-3 methyl ether, PPG-2 propyl ether, propanediol, propylene glycol, propylene glycol butyl ether, propylene glycol propyl ether, tetrahydrofuran, trimethylhexanol, phenol, benzene, toluene, xylene; as well as water, optionally in mixture with dispersion aids and also mixtures of the aforementioned substances.

Preferred solvents include, but are not limited to, one or more organic solvents from the group consisting of ethanol, isopropanol, n-propanol, dipropylene glycol methyl ether and butoxyisopropanol (1,2-propylene glycol n-butyl ether), tetrahydrofuran, phenol, benzene, toluene, xylene, preferably ethanol, isopropanol, n-propanol and/or dipropylene glycol methyl ether, in particular isopropanol and/or n-propanol.

Active ingredients with suitable dimensions may also be occluded in pores in the activated porous carbon-based coatings.

The loading with the active ingredient may be temporary, i.e., the active ingredient may be released after implantation of the medical device or the active ingredient may be permanently immobilized in or on the carbon-based layer. In this way it is possible to produce medical implants containing active ingredients with static, dynamic or a combination of static and dynamic active ingredient loads. This yields multifunctional coatings based on the carbon-based layers produced according to this invention.

In static loading with active ingredients, the active ingredients are essentially immobilized permanently in or on the coating. Active ingredients used for this purpose include, but are not limited to, inorganic substances, e.g., hydroxyl apatite (HAP), fluorapatite, tricalcium phosphate (TCP), zinc and/or organic substances such as peptides, proteins, carbohydrates such as monosaccharides, oligosaccharides and polysaccharides, lipids, phospholipids, steroids, lipoproteins, glycoproteins, glycolipids, proteoglycans, DNA, RNA, signal peptides or antibodies and/or antibody fragments, bioabsorbable polymers, e.g., polylactic acid, chitosan as well as pharmacologically active substances or substance mixtures, combinations thereof and the like.

In the case of dynamic active ingredient loading, it is provided that the applied active ingredients will be released after implantation of the medical device in the body. In this way the coated implants may be used for therapeutic purposes, with the active ingredients applied to the implant being released locally and successively at the site of use of the implant. Active ingredients that can be used in dynamic active ingredient loading for the release of active ingredients include, but are not limited to, for example, hydroxyl apatite (HAP), fluorapatite, tricalcium phosphate (TCP), zinc and/or organic substances such as peptides, proteins, carbohydrates such as monosaccharides, oligosaccharides and polysaccharides, lipids, phospholipids, steroids, lipoproteins, glycoproteins, glycolipids, proteoglycans, DNA, RNA, signal peptides or antibodies and/or antibody fragments, bioabsorbable polymers, e.g., polylactic acid, chitosan and the like as well as pharmacologically active substances or substance mixtures.

Suitable pharmacologically active substances or substance mixtures for static and/or dynamic loading of implantable medical devices coated according to this invention include, but are not limited to, active ingredients or active ingredient combinations selected from heparin, synthetic heparin analogs (e.g., fondaparinux), hirudin, antithrombin III, drotrecogin alpha; fibrinolytics such as alteplase, plasmin, lysokinases, factor XIIa, prourokinase, urokinase, anistreplase, streptokinase; platelet aggregation inhibitors such as acetylsalicylic acid [aspirin], ticlopidine, clopidogrel, abciximab, dextrans; corticosteroids such as alclometasone, amcinonide, augmented betamethasone, beclomethasone, betamethasone, budesonide, cortisone, clobetasol, clocortolone, desonide, desoximetasone, dexamethasone, fluocinolone, fluocinonide, flurandrenolide, flunisolide, fluticasone, halcinonide, halobetasol, hydrocortisone, methylprednisolone, mometasone, prednicarbate, prednisone, prednisolone, triamcinolone; so-called non-steroidal anti-inflammatory drugs (NSAIDs) such as diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, piroxicam, salsalate, sulindac, tolmetin, celecoxib, rofecoxib; cytostatics such as alkaloides and podophyllum toxins such as vinblastine, vincristine; alkylating agents such as nitrosoureas, nitrogen lost analogs; cytotoxic antibiotics such as daunorubicin, doxorubicin and other anthracyclines and related substances, bleomycin, mitomycin; antimetabolites such as folic acid analogs, purine analogs or pyrimidine analogs; paclitaxel, docetaxel, sirolimus; platinum compounds such as carboplatin, cisplatin or oxaliplatin; amsacrin, irinotecan, imatinib, topotecan, interferon-alpha 2a, interferon-alpha 2b, hydroxycarbamide, miltefosine, pentostatin, porfimer, aldesleukin, bexaroten, tretinoin; antiandrogens and antiestrogens; antiarrythmics in particular class I antiarrhythmic such as antiarrhythmics of the quinidine type, quinidine, dysopyramide, ajmaline, prajmalium bitartrate, detajmium bitartrate; antiarrhythmics of the lidocaine type, e.g., lidocaine, mexiletin, phenytoin, tocainid; class Ic antiarrhythmics, e.g., propafenon, flecainid(acetate); class II antiarrhythmics beta-receptor blockers such as metoprolol, esmolol, propranolol, metoprolol, atenolol, oxprenolol; class III antiarrhythmics such as amiodarone, sotalol; class IV antiarrhythmics such as diltiazem, verapamil, gallopamil; other antiarrhythmics such as adenosine, orciprenaline, ipratropium bromide; agents for stimulating angiogenesis in the myocardium such as vascular endothelial growth factor (VEGF), basic fibroblast growth factor (bFGF), non-viral DNA, viral DNA, endothelial growth factors: FGF-1, FGF-2, VEGF, TGF; antibiotics, monoclonal antibodies, anticalins; stem cells, endothelial progenitor cells (EPC); digitalis glycosides, such as acetyl digoxin/metildigoxin, digitoxin, digoxin; cardiac glycosides such as ouabain, proscillaridin; antihypertensives such as CNS active antiadrenergic substances, e.g., methyldopa, imidazoline receptor agonists; calcium channel blockers of the dihydropyridine type such as nifedipine, nitrendipine; ACE inhibitors: quinaprilate, cilazapril, moexipril, trandolapril, spirapril, imidapril, trandolapril; angiotensin II antagonists: candesartancilexetil, valsartan, telmisartan, olmesartanmedoxomil, eprosartan; peripherally active alpha-receptor blockers such as prazosin, urapidil, doxazosin, bunazosin, terazosin, indoramin; vasodilatators such as dihydralazine, diisopropylamine dichloracetate, minoxidil, nitroprusside sodium; other antihypertensives such as indapamide, co-dergocrine mesylate, dihydroergotoxin methanessulfonate, cicletanin, bosentan, fludrocortisone; phosphodiesterase inhibitors such as milrinon, enoximon and antihypotensives such as in particular adrenergic and dopaminergic substances such as dobutamine, epinephrine, etilefrine, norfenefrine, norepinephrine, oxilofrine, dopamine, midodrine, pholedrine, ameziniummetil; and partial adrenoceptor agonists such as dihydroergotamine; fibronectin, polylysine, ethylene vinyl acetate, inflammatory cytokines such as: TGFβ, PDGF, VEGF, bFGF, TNFα, NGF, GM-CSF, IGF-a, IL-1, IL-8, IL-6, growth hormone; as well as adhesive substances such as cyanoacrylates, beryllium, silica; and growth factors such as erythropoetin, hormones such as corticotropins, gonadotropins, somatropins, thyrotrophins, desmopressin, terlipressin, pxytocin, cetrorelix, corticorelin, leuprorelin, triptorelin, gonadorelin, ganirelix, buserelin, nafarelin, goserelin, as well as regulatory peptides such as somatostatin, octreotid; bone and cartilage stimulating peptides, bone morphogenetic proteins (BMPs), in particulary recombinant BMPs, such as recombinant human BMP-2 (rhBMP-2), bisphosphonate (e.g., risedronate, pamidronate, ibandronate, zoledronic acid, clodronsäure, etidronsäure, alendronic acid, tiludronic acid), fluorides such as disodium fluorophosphate, sodium fluoride; calcitonin, dihydrotachystyrol; growth factors and cytokines such as epidermal growth factor (EGF), platelet-derived growth factor (PDGF), fibroblast growth factors (FGFs), transforming growth factors-b (TGFs-b), transforming growth factor-a (TGF-a), erythropoietin (Epo), insulin-like growth factor-I (IGF-I), insulin-like growth factor-II (IGF-II), interleukin-1 (IL-1), interleukin-2 (IL-2), interleukin-6 (IL-6), interleukin-8 (IL-8), tumor necrosis factor-a (TNF-a), tumor necrosis factor-b (TNF-b), interferon-g (INF-g), colony stimulating factors (CSFs); monocyte chemotactic protein, fibroblast stimulating factor 1, histamine, fibrin or fibrinogen, endothelin-1, angiotensin II, collagens, bromocriptine, methysergide, methotrexate, carbon tetrachloride, thioacetamide and ethanol; as well as silver (ions), titanium dioxide, antibiotics and anti-infective drugs such as in particular β-lactam antibiotics, e.g., β-lactamase-sensitive penicillins such as benzyl penicillins (penicillin G), phenoxymethylpenicillin (penicillin V); β-lactamase-resistent penicillins such as aminopenicillins, e.g., amoxicillin, ampicillin, bacampicillin; acylaminopenicillins such as mezlocillin, piperacillin; carboxypenicillins, cephalosporins such as cefazoline, cefuroxim, cefoxitin, cefotiam, cefaclor, cefadroxil, cefalexin, loracarbef, cefixim, cefuroximaxetil, ceftibuten, cefpodoximproxetil, cefpodoximproxetil; aztreonam, ertapenem, meropenem; β-lactamase inhibitors such as sulbactam, sultamicillintosylate; tetracyclines such as doxycycline, minocycline, tetracycline, chlorotetracycline, oxytetracycline; aminoglycosides such as gentamicin, neomycin, streptomycin, tobramycin, amikacin, netilmicin, paromomycin, framycetin, spectinomycin; macrolide antibiotics such as azithromycin, clarithromycin, erythromycin, roxithromycin, spiramycin, josamycin; lincosamides such as clindamycin, lincomycin; gyrase inhibitors such as fluoroquinolones, e.g., ciprofloxacin, ofloxacin, moxifloxacin, norfloxacin, gatifloxacin, enoxacin, fleroxacin, levofloxacin; quinolones such as pipemidic acid; sulfonamides, trimethoprim, sulfadiazine, sulfalene; glycopeptide antibiotics such as vancomycin, teicoplanin; polypeptide antibiotics such as polymyxins, e.g., colistin, polymyxin-b, nitroimidazole derivates, e.g., metronidazole, tinidazole; aminoquinolones such as chloroquin, mefloquin, hydroxychloroquin; biguanids such as proguanil; quinine alkaloids and diaminopyrimidines such as pyrimethamine; amphenicols such as chloramphenicol; rifabutin, dapson, fusidic acid, fosfomycin, nifuratel, telithromycin, fusafungin, fosfomycin, pentamidine diisethionate, rifampicin, taurolidin, atovaquon, linezolid; virus static such as aciclovir, ganciclovir, famciclovir, foscarnet, inosine-(dimepranol-4-acetamidobenzoate), valganciclovir, valaciclovir, cidofovir, brivudin; antiretroviral active ingredients (nucleoside analog reverse-transcriptase inhibitors and derivatives) such as lamivudine, zalcitabine, didanosine, zidovudin, tenofovir, stavudin, abacavir; non-nucleoside analog reverse-transcriptase inhibitors: amprenavir, indinavir, saquinavir, lopinavir, ritonavir, nelfinavir; amantadine, ribavirine, zanamivir, oseltamivir and lamivudine, as well as any combinations and mixtures thereof.

Especially preferred embodiments of the present invention include, but are not limited to, coated vascular endoprostheses (intraluminal endoprostheses) such as stents, coronary stents, intravascular stents, peripheral stents and the like.

These can easily be functionalized in a biocompatible manner by the method according to this invention, so that the residual stenoses which frequently occur in percutaneous transluminal angioplasty with traditional stents can be prevented.

By immobilizing suitable active ingredients on porous carbon-based coatings, in particular paclitaxel, rapamycins or dexamethasone, the local inflammation reaction in the tissue of the vascular wall can be inhibited and/or suppressed by temporary local release of these active ingredients. The use and efficacy of such active ingredients is sufficiently well known according to the state of the art, but the usability has been limited due to the coating systems according to the state of the art, in particular because of the inadequate load capacity which leads to inadequate bioavailability, inadequate, i.e., incomplete release of these active ingredients or intolerance between the coating system and the active ingredient due to unwanted physical or chemical interactions.

In preferred embodiments of the present invention, vitreous carbon layers or composite layers with the addition of carbon black particles with layer thicknesses between 80 nm and 10 μm, pore sizes from 5 nm to 1 μm and porosities of 1% to 70% are produced and activated, preferably by introducing fillers and then removing them from the carbon layer or through the admixture of carbon black particles having a spherical or ellipsoid or rod-shaped morphology and a particle size of 10 nm to 200 nm, these particles creating a porous matrix, so that active ingredients can be accommodated in a sufficient amount. The surface of the stent implant may be increased here up to 2000 m²/m^{3.}

In preferred embodiments of this invention, by activation of the carbon-based layer, e.g., with air at an elevated temperature, the hydrophilic property of the coating can be increased, which in turn additionally increases the biocompatibility, while on the other hand making the layer better able to uptake active ingredients, in particular hydrophilic active ingredients.

In especially preferred embodiments, stents, in particular coronary stents and peripheral stents may be loaded with pharmacologically active substances or substance mixtures or with cells or cell cultures by the method according to this invention. For example, the carbon-based stent surfaces may be finished with the following active ingredients to locally suppress cell adhesion, platelet aggregation, complement activation and/or inflammatory tissue reactions or cell proliferation:

Heparin, synthetic heparin analogs (e.g., fondaparinux), hirudin, antithrombin III, drotrecogin alpha, fibrinolytics (alteplase, plasmin, lysokinases, factor XIIa, prourokinase, urokinase, anistreplase, streptokinase), platelet aggregation inhibitors (acetylsalicylic acid [aspirin], ticlopidine, clopidogrel, abciximab, dextran), corticosteroids (alclometasone, amcinonide, augmented betamethasone, beclomethasone, betamethasone, budesonide, cortisone, clobetasol, clocortolone, desonide, desoximetasone, dexamethasone, fluocinolone, fluocinonide, flurandrenolide, flunisolide, fluticasone, halcinonide, halobetasol, hydrocortisone, methylprednisolone, mometasone, prednicarbate, prednisone, prednisolone, triamcinolone), so-called non-steroidal anti-inflammatory drugs [NSAIDs] (diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, piroxicam, salsalate, sulindac, tolmetin, celecoxib, rofecoxib), cytostatics (alkaloids and podophyllum toxins such as vinblastine, vincristine; alkylating agents such as nitrosoureas, nitrogen lost analogs; cytotoxic antibiotics such as daunorubicin, doxorubicin and other anthracyclines and related substances, bleomycin, mitomycin; antimetabolites such as folic acid, purine or pyrimidine analogs; paclitaxel, docetaxel, sirolimus; platinum compounds such as carboplatin, cisplatin or oxaliplatin; amsacrin, irinotecan, imatinib, topotecan, interferon-alpha 2a, interferon-alpha 2b, hydroxycarbamide, miltefosine, pentostatin, porfimer, aldesleukin, bexaroten, tretinoin; antiandrogens and antiestrogens).

Stents activated according to this invention may be loaded with any of the following for their systemic cardiological effects:

Antiarrythmics, in particular class I antiarrhythmics (antiarrhythmics of the quinidine type: quinidine, dysopyramide, ajmaline, prajmalium bitartrate, detajmium bitartrate; antiarrhythmics of the lidocaine type: lidocaine, mexiletin, phenytoin, tocainid; class IC antiarrhythmics: propafenon, flecainide (acetate)), class II antiarrhythmics (beta-receptor blockers) (metoprolol, esmolol, propranolol, metoprolol, atenolol, oxprenolol), class III antiarrhythmics (amiodaron, sotalol), calss IV antiarrhythmics (diltiazem, verapamil, gallopamil), other antiarrhythmics such as adenosine, orciprenaline, ipratropium bromide; stimulation of angiogenesis in the myocardium: vascular endothelial growth factor (VEGF), basic fibroblast growth factor (bFGF), nonviral DNA, viral DNA, endothelial growth factors: FGF-1, FGF-2, VEGF, TGF; antibodies, monoclonal antibodies, anticalins; stem cells, endothelial progenitor cells (EPC). Other cardiac agents include, but are not limited to,: digitalis glycosides (acetyldigoxin/metildigoxin, digitoxin, digoxin), other cardiac glycosides (ouabain, proscillaridin). In addition the stents may be loaded antihypertensives (CNS active antiadrenergic substances; methyldopa, imidazoline receptor agonists; calcium channel blocker of the dihydropyridine type such as nifedipine, nitrendipine; ACE inhibitors: quinaprilat, cilazapril, moexipril, trandolapril, spirapril, imidapril, trandolapril; angiotensin II antagonists: candesartancilexetil, valsartan, telmisartan, olmesartanmedoxomil, eprosartan; peripheral acting alpha-receptor blockers: prazosin, urapidil, doxazosin, bunazosin, terazosin, indoramin; vasodilators: dihydralazine, diisopropylamine dichloracetate, minoxidil, nitroprusside sodium), other antihypertensives such as indapamide, co-dergocrine mesylate, dihydroergotoxin methane sulfonate, cicletanin, bosentan. In addition, phosphodiesterase inhibitors (milrinon, enoximon) and antihypotensives, here in particular adrenergic and dopaminergic substances (dobutamine, epinephrine, etilefrine, norfenefrine, norepinephrine, oxilofrine, dopamine, midodrine, pholedrine, amezinium metil), partial adrenoceptor agonists (dihydroergotamine), and finally other antihypotensives such as fludrocortisone may also be used.

To increase tissue adhesion, in particular in the case of peripheral stents, components of the extracellular matrix, fibronectin, polylysine, ethylene vinyl acetate, inflammatory cytokines such as: TGFβ, PDGF, VEGF, bFGF, TNFα, NGF, GM-CSF, IGF-a, IL-1, IL-8, IL-6, growth hormones and adhesive substances such as cyanoacrylates, beryllium or silica may also be used.

Other substances suitable for here and having a systemic and/or local effect include, but are not limited to, growth factors and erythropoetin.

Hormones may also be provided in the stent coatings such as corticotropins, gonadotropins, somatropin, thyrotrophin, desmopressin, terlipressin, oxytocin, cetrorelix, corticorelin, leuprorelin, triptorelin, gonadorelin, ganirelix, buserelin, nafarelin, goserelin, and regulatory peptides such as somatostatin and/or octreotid.

Other embodiments provide for functionalization by loading the carbon surfaces with cells, e.g., with pluripotent stem cells, endothelial cells or connective tissue cells which may be obtained from organisms, cultured from cell cultures in the laboratory or modified by genetic engineering.

For example in a special embodiment, vascular implants provided with activated carbon layers may be loaded with endothelial cell cultures by first using them as a substrate in a bioreactor and/or as a culturing and carrier system for cell cultures. Suitable methods here include, but are not limited to, those described in German Patent DE 103 35 131, i.e., PCT/EP04/00077, the disclosure content is herewith included.

For example, inventive nanoporous activated carbon layers with a surface area of 200 to 3000 m²/m³ can be loaded with endothelial cells after culturing, in which case the possible cell densities range from 10¹ to 10¹⁶ cells/mL layer volume, preferably 10³ to 10¹² cells/mL.

In the case of surgical and orthopedic implants, it may be advantageous to activate the implants with one or more carbon-based layers so that the layers are macroporous. Suitable pore sizes are in the range of 0.1 to 1000 μm, preferably 1 to 400 μm to support better integration of the implants by incorporation into the surrounding cell tissue or bone tissue.

For orthopedic and nonorthopedic implants as well as heart valves or synthetic heart parts functionalized according to this invention, if they are to be loaded with active ingredients, the same active ingredients may be used as those listed for the stent applications described above or local suppression of cell adhesion, platelet aggregation, complement activation and/or inflammatory tissue reaction or cell proliferation.

Furthermore, for stimulation of tissue growth in particular in the case of orthopedic implants, the following active ingredients may be used to improve implant integration: bone and cartilage stimulating peptides, bone morphogenetic proteins (BMPs), in particular recombinant BMPs (e.g., recombinant human BMP-2 (rhBMP-2)), bisphosphonates (e.g., risedronate, pamidronate, ibandronate, zoledronic acid, clodronic acid, etidronic acid, alendronic acid, tiludronic acid), fluorides (disodium fluorophosphate, sodium fluoride); calcitonin, dihydrotachystyrene. Then all the growth factors and cytokines may be used (epidermal growth factor (EGF), platelet-derived growth factor (PDGF), fibroblast growth factors (FGFs), transforming growth factors-b (TGFs-b), transforming growth factor-a (TGF-a), erythropoietin (Epo), insulin-like growth factor I (IGF-I), insulin-like growth factor II (IGF-II), interleukin-1 (IL-1), interleukin-2 (IL-2), interleukin-6 (IL-6), interleukin-8 (IL-8), tumor necrosis factor-a (TNF-a), tumor necrosis factor-b (TNF-b), Interferon-g (INF-g), colony stimulating factors (CSFs)). In addition to the inflammatory cytokines mentioned above, other adhesion-promoting and integration-promoting substances include, but are not limited to, the monocyte chemotactic protein, fibroblast stimulating factor 1, histamine, fibrin or fibrinogen, endothelin-1, angiotensin II, collagens, bromocriptine, methysergide, methotrexate, carbon tetrachloride, thioacetamide, ethanol.

In addition, the implants, stents and the like that have been activated according to this invention may also be provided with antibacterial-antiinfectious coatings or impregnation instead of or in addition to pharmaceutical drugs, in which case the following substances or substance mixtures may be used: silver (ions), titanium dioxide, antibiotics and antiinfective agents. In particular these include, but are not limited to, beta-lactam antibiotics (β-lactam antibiotics: β-lactamase-sensitive penicillins such as benzyl penicillins (penicillin G), phenoxymethyl penicillin (penicillin V)); β-lactamase-resistant penicillins such as aminopenicillins, e.g., amoxicillin, ampicillin, bacampicillin; acylaminopenicillins such as mezlocillin, piperacillin; carboxypenicillins, cephalosporins (cefazolin, cefuroxim, cefoxitin, cefotiam, cefaclor, cefadroxil, cefalexin, loracarbef, cefixim, cefuroximaxetil, ceftibuten, cefpodoximproxetil, cefpodoximproxetil) or other such as aztreonam, ertapenem, meropenem. Other antibiotics that may be used include, but are not limited to, β-lactamase inhibitors (sulbactam, sultamicillintosylate), tetracyclins (doxycyclin, minocyclin, tetracyclin, chlortetracyclin, oxytetracyclin), aminoglycosides (gentamicin, neomycin, streptomycin, tobramycin, amikacin, netilmicin, paromomycin, framycetin, spectinomycin), macrolide antibiotics (azithromycin, clarithromycin, erythromycin, roxithromycin, spiramycin, josamycin), lincosamids (clindamycin, lincomycin), gyrase inhibitors (fluoroquinolones such as ciprofloxacin, ofloxacin, moxifloxacin, norfloxacin, gatifloxacin, enoxacin, fleroxacin, levofloxacin; other quinolones such as pipemidic acid), sulfonamids and trimethoprim (sulfadiazin, sulfalen, trimethoprim), glycopeptide antibiotics (vancomycin, teicoplanin), polypeptide antibiotics (polymyxins such as colistin, polymyxin-b), nitroimidazole derivates (metronidazole, tinidazole), aminoquinolones (chloroquin, mefloquin, hydroxychloroquin), biguanids (proguanil), quinine alkaloids and diaminopyrimidines (pyrimethamine), amphenicols (chloramphenicol) and other antibiotics (rifabutin, dapson, fusidinic acid, fosfomycin, nifuratel, telithromycin, fusafungin, fosfomycin, pentamidine diisethionate, rifampicin, taurolidine, atovaquone, linezolide). Examples of virus statics that can be mentioned include, but are not limited to, aciclovir, ganciclovir, famciclovir, foscarnet, inosine-(dimepranol-4-acetamidobenzoate), valganciclovir, valaciclovir, cidofovir, brivudin. This also include, but are not limited to, antiretroviral active ingredients (nucleoside analog reverse-transcriptase inhibitors and derivatives: lamivudine, zalcitabine, didanosine, zidovudin, tenofovir, stavudin, abacavir; non-nucleoside analog reverse-transcriptase inhibitors: amprenavir, indinavir, saquinavir, lopinavir, ritonavir, nelfinavir) and other virustatics such as amantadin, ribavirin, zanamivir, oseltamivir, lamivudin.

In especially preferred embodiments of the present invention, the inventive implants may be suitably modified in their chemical or physical properties with carbon-based layers before or after loading of the active ingredient by using other agents, e.g., to modify the hydrophilicity, hydrophobicity, electric conductivity, adhesion or other surface properties. Substances that can be used for this purpose include, but are not limited to, biodegradable or non-degradable polymers such as for the biodegradable polymers: collagens, albumin, gelatin, hyaluronic acid, starch, cellulose (methyl cellulose, hydroxypropyl cellulose, hydroxypropylmethyl cellulose, carboxymethyl cellulose phthalate; also casein, dextrans, polysaccharides, fibrinogen, poly(D,L-lactides), poly(D,L-lactide-co-glycolide), poly(glycolides), poly(hydroxybutylates), poly(alkyl carbonates), poly(ortho esters), polyesters, poly(hydroxyvaleric acid), polydioxanones, poly(ethylene terephtalate), poly(malic acid), poly(tartronic acid), polyanhydride, polyphosphohazenes, poly(amino acids) and all their copolymers.

The non-biodegradable polymers include, but are not limited to,: poly(ethylene vinyl acetates), silicones, acrylic polymers such as polyacrylic acid, polymethyl acrylic acid, polyacryl cyanoacrylate; polyethylenes, polypropylenes, polyamides, polyurethanes, poly(ester urethanes), poly(ether urethanes), poly(ester ureas), polyethers such as polyethylene oxide, polypropylene oxide, pluronics, polytetramethylene glycol; vinyl polymers such as polyvinylpyrrolidones, poly(vinyl alcohols), poly(vinyl acetate phthalate); parylenes.

It is true in general that polymers with anionic properties (e.g., alginate, carrageenan, carboxymethyl cellulose) or cationic (e.g., chitosan, poly-L-lysine, etc.) or both properties (phosphorylcholine) can be produced.

These polymers can be applied to the surface of the implants and may cover them partially or entirely.

To modify the release properties of inventive implants containing one or more active ingredients, specific pH-dependent or temperature-dependent release properties can be achieved by applying additional polymers, for example. Examples of pH-sensitive polymers include, but are not limited to, poly(acrylic acid) and derivatives thereof, e.g., homopolymers such as poly(aminocarboxylic acid), poly(acrylic acid), poly(methyl acrylic acid) and copolymers thereof. This is also true of polysaccharides such as cellulose acetate phthalate, hydroxypropylmethyl cellulose phthalate, hydroxypropylmethyl cellulose succinate, cellulose acetate trimellitate and chitosan. Thermally sensitive polymers include, but are not limited to, for example poly(N-isopropylacrylamide-co-sodium acrylate-co-n-N-alkylacrylamide), poly(N-methyl-N-n-propylacrylamide), poly(N-methyl-N-isopropylacrylamide), poly(N-n-propylmethacrylamide), poly(N-isopropylacrylamide), poly(N,n-diethylacrylamide), poly(N-isopropylmethacrylamide), poly(N-cyclopropylacrylamide), poly(N-ethyl-acrylamide), poly(N-ethyl-methyacrylamide), poly(N-methyl-N-ethylacrylamide), poly(N-cyclopropylacrylamide). Other polymers with thermogel characteristics include, but are not limited to, hydroxypropyl cellulose, methyl cellulose, hydroxypropylmethyl cellulose, ethylhydroxyethyl cellulose and pluronics such as F-127, L-122, L-92, L-81, L-61.

The active ingredients may be adsorbed in the pores of the carbon-based layer (covalently, non-covalently) in which case their release can be controlled primarily through pore size and pore geometry. Additional modifications of the porous chemical layer by chemical modification (anionic, cationic) make it possible to modify the release, e.g., as a function of pH. The release of carriers that contain active ingredient also constitutes another application namely microcapsules, liposomes, nanocapsules, nanoparticles, micelles, synthetic phospholipids, gas dispersions, emulsions, microemulsions, nanospheres etc., which are adsorbed in the pores of the carbon layer and are then released therapeutically. By additional covalent or non-covalent modification of the carbon layer, the pores can be occluded so that bioactive ingredients are protected. Possibilities include, but are not limited to, the above-mentioned polysaccharides, lipids, etc., but also the polymers mentioned above.

Therefore, in the additional coating of the porous carbon-based layers produced according to this invention with additional layers, a distinction may be made between physical barriers such as inert biodegradable substances (poly-L-lysine, fibronectin, chitosan, heparin, etc.) and biologically active barriers. The latter may be sterically hindering molecules which are physiologically bioactivated and which permit the release of active ingredients and/or their vehicles. Examples include, but are not limited to, enzymes which mediate the release, activate biologically active substances or bind inactive coatings and lead to exposure of active ingredients. All this specific mechanisms and properties listed here can be applied to the primary carbon layer as well as additional layers applied thereto.

By applying the release-modifying polymer layers mentioned above and/or by adapting the pore structure of the carbon-based layer, it is possible to control the release of the active ingredients from the implant in a wide range. achievable release times include, but are not limited to, up to twelve hours, up to one or more years, preferably 24 hours, 48 hours, 96 hours, one week, two weeks, one month, three months.

The inventive implants may also be loaded with live cells or microorganisms in special applications and may be functionalized in this way. These cells or microorganisms may form colonies in suitably porous carbon-based layers and the implant colonized in this way may be provided with a suitable membrane coating which is permeable for nutrients and active ingredients produced by cells or microorganisms but is not permeable for the cells themselves. Thus the cells or microorganisms can be supplied from the organism through the membrane coating.

For example, by using the inventive technology in this way, it is possible to produce implants which contain insulin-producing cells that produce and release insulin according to the prevailing glucose level after being implanted in the body.

The invention is further described by the following numbered paragraphs:

1. A method for producing medical implants having functionalized surfaces comprising the following steps:
a) providing a medical implant with at least one carbon-based layer on at least part of the surface of the implant;
b) activating the carbon-based layer by creating porosity;
c) functionalizing the activated carbon-based layer.

2. The method according to paragraph 1, characterized in that the carbon-based layer is selected from pyrolytically produced carbon, vapor-deposited carbon, carbon applied by CVD, PVD or sputtering, metal carbides, metal carbonitrides, metal oxynitrides or metal oxycarbides as well as any desired combinations thereof.

3. The method according to paragraph 1 or 2, characterized in that the implant consists of a material which is selected from carbon, carbon composite material, carbon fibers, ceramic, glass, plastics, metals, alloys, bone, stone or minerals.

4. The method according to any one of the preceding paragraphs, characterized in that the implant is selected from medical or therapeutic implants such as vascular endoprostheses, stents, coronary stents, peripheral stents, surgical or orthopedic implants, bone prostheses or joint prostheses, artificial hearts, artificial heart valves, subcutaneous and/or intramuscular implants.

5. The method according to any one of the preceding paragraphs, characterized in that activation of the carbon-based layer is performed with suitable oxidizing agents and/or reducing agents.

6. The method according to any one of the preceding paragraphs, characterized in that the carbon-based layer is activated by oxidation with air, oxygen, nitrous oxide, and/or oxidizing acids, optionally at an elevated temperature.

7. The method according to any one of the preceding paragraphs, characterized in that the activation is performed by abrasion in an aqueous ultrasonic bath with the addition of alumina, silicates and/or aluminates.

8. The method according to any one of the preceding paragraphs, characterized in that activation causes the carbon-based layer to become porous, preferably macroporous with pore diameters in the range of 0.1 to 1000 mm, optionally also by prestructuring the substrate.

9. The method according to any one of the preceding paragraphs, characterized in that activation causes the carbon-based layer to become nanoporous.

10. The method according to any one of the preceding paragraphs, characterized in that the activate porous carbon-based layer is subsequently compressed and/or sealed by CVD and/or CVI of volatile organic substances.

11. The method according to any one of the preceding paragraphs, characterized in that the functionalization of the activated carbon-based layer comprises loading the layer with at least one substance selected from pharmacological active ingredients, linkers, microorganisms, plant or animal cells including human cells or cell cultures and tissue, minerals, salts, metals, synthetic or natural polymers, proteins, peptides, amino acids, solvents, ions, cations, in particular metal cations such as cobalt, nickel, copper, zinc cations, antibodies, calmodulin, chitin, cellulose, sugars, amino acids, glutathione, streptavidin, Strep-Tactin or other mutants or S protein, dextrans, as well as their derivatives, mixtures and combinations.

12. The method according to any one of the preceding paragraphs, characterized in that the functionalization is performed by adsorption of substances corresponding to affinity tags in and/or on the carbon-based layer, whereby the corresponding substances are selected so that they can enter into a bond with the affinity tags.

13. The method according to paragraph 11 or 12, characterized in that the substance(s) is/are applied to and/or immobilized on the carbon-based layer by adsorption, absorption, physisorption, chemisorption, electrostatic covalent bonding or non-covalent bonding.

14. The method according to paragraph 11, characterized in that the at least one substance is essentially permanently immobilized on the carbon-based layer(s).

15. The method according to paragraph 11, characterized in that the at least one substance applied to the carbon-based layer, in particular a pharmacological active ingredient, can be released from the layer in a controlled manner.

16. The method according to paragraph 15, characterized in that the pharmacologically active substances are incorporated into microcapsules, liposomes, nanocapsules, nanoparticles, micelles, synthetic phospholipids, gas dispersions, emulsions, microemulsions or nanospheres which are adsorbed in the pores or on the surface of the carbon-based layer and can then be released therapeutically.

17. The method according to any one of paragraphs 14 through 16, characterized in that the a coating which influences the release of the active ingredient is also applied, selected from pH-sensitive and/or temperature-sensitive polymers and/or biologically active barriers such as enzymes.

18. The method according to any one of the preceding paragraphs, characterized in that the functionalization includes applying biodegradable and/or absorbable polymers such as collagen, albumin, gelatin, hyaluronic acid, starch, celluloses such as methyl cellulose hydroxypropyl cellulose, hydroxypropylmethyl cellulose, carboxymethylcellulose phthalate; casein, dextrans, polysaccharides, fibrinogen, poly(D,L-lactide), poly(D,L-lactide-co-glycolide), poly(glycolide), poly(hydroxybutylate), poly(alkyl carbonate), poly(orthoester), polyester, poly(hydroxyvaleric acid), polydioxanone, poly(ethylene terephtalate), poly(malic acid), poly(tartronic acid), polyanhydrides, polyphosphazenes, poly(amino acids) and their copolymers.

19. The method according to any one of the preceding paragraphs, characterized in that the functionalization includes applying non-biodegradable and/or non-absorbable polymers such as poly(ethylene vinyl acetate), silicones, acrylic polymers such as polyacrylic acid, polymethyl acrylic acid, polyacryl cyanoacrylate; polyethylenes, polypropylenes, polyamides, polyurethanes, poly(ester urethanes), poly(ether urethanes), poly(ester ureas), polyethers, polyethylene oxide, polypropylene oxide, pluronics, polytetramethylene glycol; vinyl polymers such as polyvinylpyrrolidones, poly(vinyl alcohols), poly(vinyl acetate phthalate) as well as their copolymers.

20. An implant having a functionalized surface producible according to any one of the preceding paragraphs.

21. The implants according to paragraph 20, characterized in that it is made of metals such as stainless steel, titanium, tantalum, platinum, gold, palladium, alloys, in particular memory alloys such as nitinol or nickel titanium alloys or carbon fibers, solid carbon material or carbon composites.

22. The implant according to any one of paragraphs 20 or 21 comprising multiple carbon-based layers optionally loaded with active ingredient.

23. A device according to any one of paragraphs 20 though 22, also comprising anionic or cationic or amphoteric coatings selected from alginate, carrageenan, carboxymethyl cellulose, poly(meth)acrylates, chitosan, poly-L-lysines and/or phosphorylcholine.

24. A stent coated with an active ingredient according to any one of paragraphs 20 through 23.

25. A heart valve coated with an active ingredient according to any one of paragraphs 20 through 23.

26. The implant according to any one of paragraphs 20 through 23 in the form of an orthopedic bone prosthesis or joint prosthesis, a bone substitute or a vertebral substitute in the thoracic or lumbar region of the spinal column.

27. An active ingredient depot with controlled release that can be used subcutaneously and/or intramuscularly according to any one of paragraphs 20 through 23.

28. The implant according to any one of paragraphs 20 through 27, comprising applied and/or incorporated microorganisms, viral vectors or cells or tissue.

29. A use of an implant according to paragraph 28 for producing a therapeutic effect or for increasing the bioavailability of the implant after implantation of the implant in the human body.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A method for producing medical implants having functionalized surfaces comprising the following steps:
a) providing a medical implant with at least one carbon-based layer on at least part of the surface of the implant;
b) activating the carbon-based layer by creating porosity;
c) functionalizing the activated carbon-based layer.

2. The method according to claim 1 wherein the carbon-based layer is selected from pyrolytically produced carbon, vapor-deposited carbon, carbon applied by CVD, PVD or sputtering, metal carbides, metal carbonitrides, metal oxynitrides or metal oxycarbides as well as any desired combinations thereof.

3. The method according to claim 1 wherein the implant consists of a material which is selected from carbon, carbon composite material, carbon fibers, ceramic, glass, plastics, metals, alloys, bone, stone or minerals.

4. The method according to claim 1 wherein the implant is selected from medical or therapeutic implants such as vascular endoprostheses, stents, coronary stents, peripheral stents, surgical or orthopedic implants, bone prostheses or joint prostheses, artificial hearts, artificial heart valves, subcutaneous and/or intramuscular implants.

5. The method according to claim 1 wherein activation of the carbon-based layer is performed with suitable oxidizing agents and/or reducing agents.

6. The method according to claim 1 wherein the carbon-based layer is activated by oxidation with air, oxygen, nitrous oxide, and/or oxidizing acids, optionally at an elevated temperature.

7. The method according to claim 1 wherein the activation is performed by abrasion in an aqueous ultrasonic bath with the addition of alumina, silicates and/or aluminates.

8. The method according to claim 1 wherein activation causes the carbon-based layer to become porous, preferably macroporous with pore diameters in the range of 0.1 to 1000 mm, optionally also by prestructuring the substrate.

9. The method according to claim 1 wherein activation causes the carbon-based layer to become nanoporous.

10. The method according to claim 1 wherein the activated porous carbon-based layer is subsequently compressed and/or sealed by CVD and/or CVI of volatile organic substances.

11. The method according to claim 1 wherein the functionalization of the activated carbon-based layer comprises loading the layer with at least one substance selected from pharmacological active ingredients, linkers, microorganisms, plant or animal cells including human cells or cell cultures and tissue, minerals, salts, metals, synthetic or natural polymers, proteins, peptides, amino acids, solvents, ions, cations, in particular metal cations such as cobalt, nickel, copper, zinc cations, antibodies, calmodulin, chitin, cellulose, sugars, amino acids, glutathione, streptavidin, Strep-Tactin or other mutants or S protein, dextrans, as well as their derivatives, mixtures and combinations.

12. The method according to claim 1 wherein the functionalization is performed by adsorption of substances corresponding to affinity tags in and/or on the carbon-based layer, whereby the corresponding substances are selected so that they can enter into a bond with the affinity tags.

13. The method according to claim 11 or 12 wherein the substance(s) is/are applied to and/or immobilized on the carbon-based layer by adsorption, absorption, physisorption, chemisorption, electrostatic covalent bonding or non-covalent bonding.

14. The method according to claim 11 wherein the at least one substance is essentially permanently immobilized on the carbon-based layer(s).

15. The method according to claim 11 wherein the at least one substance applied to the carbon-based layer, in particular a pharmacological active ingredient, can be released from the layer in a controlled manner.

16. The method according to claim 15 wherein the pharmacologically active substances are incorporated into microcapsules, liposomes, nanocapsules, nanoparticles, micelles, synthetic phospholipids, gas dispersions, emulsions, microemulsions or nanospheres which are adsorbed in the pores or on the surface of the carbon-based layer and can then be released therapeutically.

17. The method according to claim 14 or 15 wherein a coating which influences the release of the active ingredient is also applied, selected from pH-sensitive and/or temperature-sensitive polymers and/or biologically active barriers such as enzymes.

18. The method according to claim 1 wherein the functionalization includes applying biodegradable and/or absorbable polymers such as collagen, albumin, gelatin, hyaluronic acid, starch, celluloses such as methyl cellulose hydroxypropyl cellulose, hydroxypropylmethyl cellulose, carboxymethylcellulose phthalate; casein, dextrans, polysaccharides, fibrinogen, poly(D,L-lactide), poly(D,L-lactide-co-glycolide), poly(glycolide), poly(hydroxybutylate), poly(alkyl carbonate), poly(orthoester), polyester, poly(hydroxyvaleric acid), polydioxanone, poly(ethylene terephtalate), poly(malic acid), poly(tartronic acid), polyanhydrides, polyphosphazenes, poly(amino acids) and their copolymers.

19. The method according to claim 1 wherein the functionalization includes applying non-biodegradable and/or non-absorbable polymers such as poly(ethylene vinyl acetate), silicones, acrylic polymers such as polyacrylic acid, polymethyl acrylic acid, polyacryl cyanoacrylate; polyethylenes, polypropylenes, polyamides, polyurethanes, poly(ester urethanes), poly(ether urethanes), poly(ester ureas), polyethers, polyethylene oxide, polypropylene oxide, pluronics, polytetramethylene glycol; vinyl polymers such as polyvinylpyrrolidones, poly(vinyl alcohols), poly(vinyl acetate phthalate) as well as their copolymers.

20. An implant having a functionalized surface produced according to the method of claim 1.

21. The implant according to claim 20 wherein the implant is made of metals such as stainless steel, titanium, tantalum, platinum, gold, palladium, alloys, in particular memory alloys such as nitinol or nickel titanium alloys or carbon fibers, solid carbon material or carbon composites.

22. The implant according to claim 20 further comprising multiple carbon-based layers optionally loaded with active ingredient.

23. A device according to claim 20 further comprising anionic or cationic or amphoteric coatings selected from alginate, carrageenan, carboxymethyl cellulose, poly(meth)acrylates, chitosan, poly-L-lysines and/or phosphorylcholine.

24. A stent coated with an active ingredient according to claim 20.

25. A heart valve coated with an active ingredient according to claim 20.

26. The implant according to claim 20 in the form of an orthopedic bone prosthesis or joint prosthesis, a bone substitute or a vertebral substitute in the thoracic or lumbar region of the spinal column.

27. An active ingredient depot with controlled release that can be used subcutaneously and/or intramuscularly having a functionalized surface produced according to the method of claim 1.

28. The implant according to claim 20 further comprising applied and/or incorporated microorganisms, viral vectors or cells or tissue.

29. A use of an implant of claim 28 for producing a therapeutic effect or for increasing the bioavailability of the implant after implantation of the implant in the human body.
